(19) United States
(12) Patent Application Publication
(10) Pub. No.: US 2008/0045950 A1
(43) Pub. Date: Feb. 21, 2008

(54) Reducing device

(75) Inventor: Jonathan M. Dewey, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: Warsaw Orthopedic, Inc.

(21) Appl. No.: 11/505,508

(22) Filed: Aug. 17, 2006

(51) Int. Cl. A61F 2/30

(52) U.S. Cl. 606/61

(57) ABSTRACT

A device for reducing a stabilization member into the head of a medical implant is provided. The device includes a first jaw for engaging the implant underneath the implant head. A second jaw is provided for engaging the stabilization number. The second jaw is slideably mounted with respect to the first jaw and is movable with respect to the first jaw to reduce the stabilization member within the head of the implant

### FIELD OF THE INVENTION

The present invention relates to reducing devices used for reducing stabilization members such as rods or plates into medical implants such as pedicle screws or lamina hooks.

### BACKGROUND OF THE INVENTION

Spinal surgeons often treat spinal disorders with spinal fusion augmented with stabilization members such as plates or elongated spinal rods connected to the spine with implants such as pedicle screws or lamina hooks. Such “stabilization assemblies” often comprise one or two spinal rods or plates and a plurality of implants engaging the pedicles of their respective vertebral bodies. The implants are provided with U-shaped heads that can be capped to couple the stabilization members to the implants or with threaded heads that can be threadably connected to the stabilization members with fasteners. During the surgical procedure to couple the stabilization member to the implants, it is often necessary to use a tool or device to push or “reduce” the stabilization member with the implant heads. It is desirable that such a device offers the surgeon a significant mechanical advantage in the reduction procedure.

### SUMMARY OF THE INVENTION

To make manifest the above noted and other manifold desires, a revelation of the present invention is brought forth The present invention provides a device for reducing a stabilization member into the head of a medical implant. The device includes a first jaw for engaging the implant underneath the implant head. A second jaw is provided for engaging the stabilization number. The second jaw is slideably mounted with respect to the first jaw and is movable with respect to the first jaw to reduce the stabilization member with the head of the implant.

Other features of the invention will become more apparent to those skilled in the art as the invention is further revealed in the accompanying drawings and Detailed Description of the Invention.

### BRIEF DESCRIPTION OF THE DRAWINGS

FIG. 1 is a schematic perspective view of a preferred embodiment reducing device of the present invention being utilized with a pedicle screw and a rod implant.

FIG. 1A is a schematic side elevational view of the reducing device of FIG. 1 being utilized with a lamina hook and a rod implant

FIG. 2 is a schematic side elevational view of the reducing device of FIG. 1.

FIG. 3 is a schematic perspective view of a preferred embodiment reducing device of the present invention being utilized with a pedicle screw and a plate implant.

FIG. 4 is a schematic side elevational view of the reducing device shown in FIG. 3.

FIG. 5 is a schematic side elevational view of an embodiment of the present invention.

FIG. 6 is a view taken along line 66 of FIG. 5.

FIG. 7 is a schematic side elevational view of an embodiment of the present invention.

FIG. 8 is a schematic side elevational view of an embodiment of the present invention.

FIG. 9 is a schematic side elevational view of an embodiment of the present invention.

FIG. 10 is a schematic side elevational view of an embodiment of the present invention.

FIG. 11 is a schematic perspective view of an alternate embodiment first jaw of the present invention reducing device.

### DETAILED DESCRIPTION OF THE INVENTION

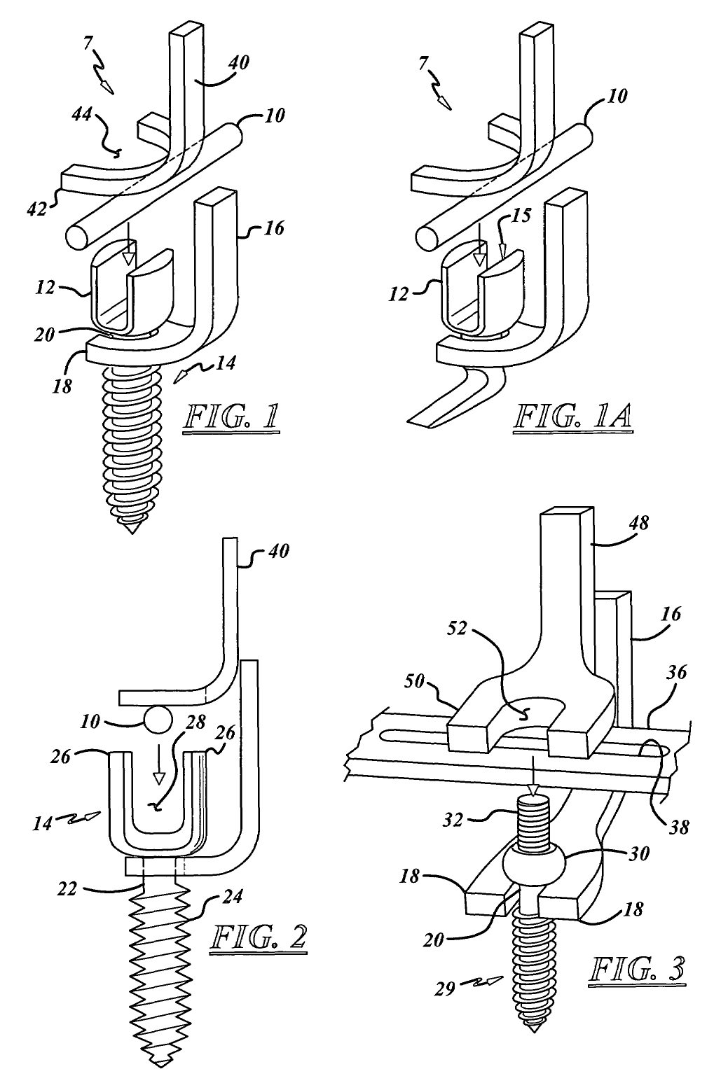

FIGS. 1 and 2 schematically illustrate a device 7 for reducing a stabilization member 10 with the head 12 of an implant pedicle screw 14. FIG. 1A schematically illustrates a device 7 for reducing a stabilization member 10 with the head 12 of an implant lamina hook 15. The device 7 has a first jaw 16. The first jaw 16 has two arms 18 forming an opening or slot 20 (FIG. 3) therebetween. The first jaw 16 engages the pedicle screw 14 underneath its head 12 on a stem portion 22 of the pedicle screw or upon a threaded portion 24. In a similar manner, the first jaw 16 engages the lamina hook 15 underneath its head 12.

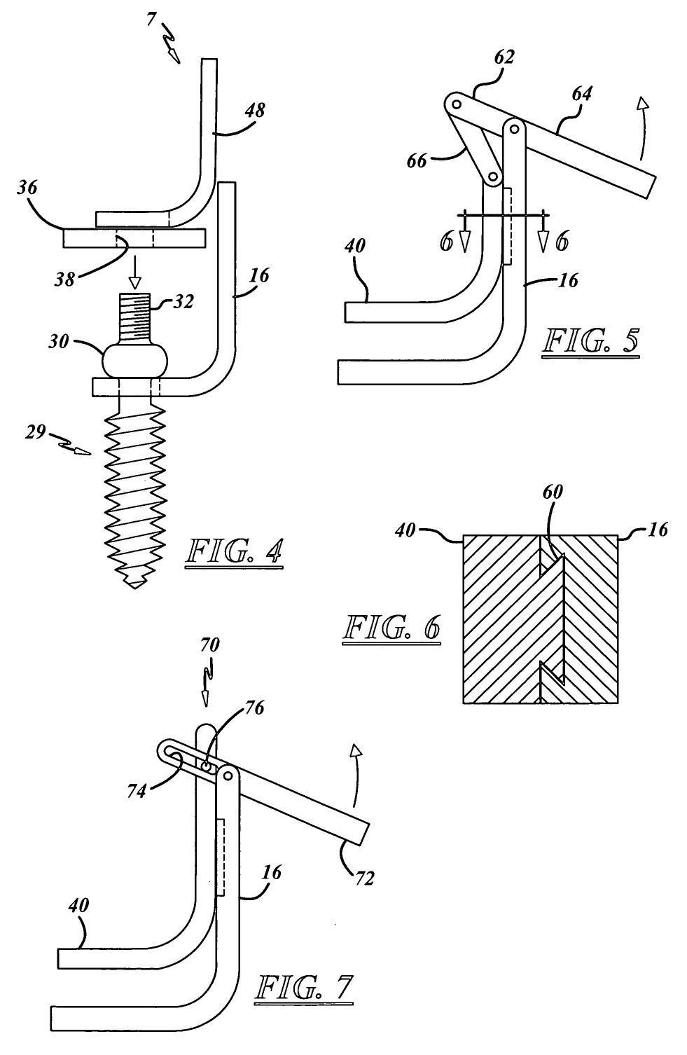

An implant 14 utilized with the reducing device 7 of the present invention can have a head 12 with two posts 26 juxtaposed by a stabilization member receiving section 28. In other applications the implant 29 can have a head providing by an enlargement 30 (FIG. 3) crowned by a threaded post 32. A stabilization member can be an elongated member such as a cable or rod 10 (FIGS. 1 and 2) or a plate 36 preferably having a slot 38 (FIGS. 3 and 4).

Slideably mounted with respect to the first jaw 16 is a second jaw 40. The second jaw 40 as shown in FIGS. 1 and 2 has two widely spaced apart arms 42 with a slot or opening 44 therebetween to allow the arms to be juxtaposed by the implant head 12. The second jaw 48 as shown in FIGS. 3 and 4 have two arms 50 with a more narrow opening or slot 52 there between to more closely align with the juxtaposing threaded post 32.

In the example shown in FIGS. 5 and 6, the second jaw 40 has a tongue and groove type connection 60 with the first jaw 16. A three pivot point lever mechanism 62 is provided to move the second jaw 40 with respect to the first jaw 16. The lever mechanism 62 includes a two bar linkage provided by a handle 64 and pivot link 66.

Referring to FIG. 7, a lever mechanism 70 has a pin and slot arrangement to move the second jaw 40 with respect to the first jaw 16. A handle 72 has a slot 74 capturing a pin 76. The pin 76 is fixedly connected with a second jaw 40. In another embodiment (not shown), the pin can be connected with the handle 72 and a slot can be provided in the second jaw 40.

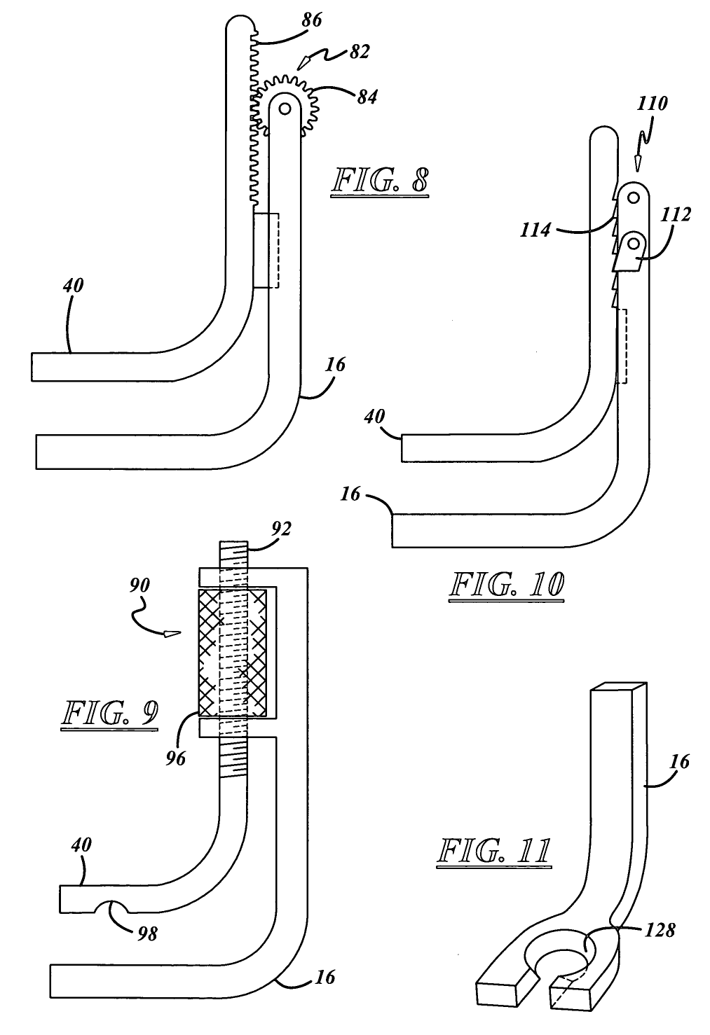

Referring to FIG. 8, the rack and pinion mechanism 82 is used to move the second jaw 40 with respect to the first jaw 16. The pinion 84 can be turned by hand or it can be motorized. The pinion 84 engages a rack 86 connected with the second jaw 40. In another embodiment (not shown), the pinion can be connected with the second jaw 40 and the rack can be connected with the first jaw 16.

Referring to FIG. 9, a jackscrew mechanism 90 is utilized to move the second jaw 40 with respect to the first jaw 16. A threaded cylinder 92 provides an upper portion of the second jaw. The cylinder 92 is encircled by an internally threaded drum 96 that can be rotated to translate the second jaw 40. Additionally the second jaw 40 has an indention 98 to provide a better grip of the stabilization number such as a rod 10 or a portion of the implant head. A similar or identical indention can also be used on the second jaw of the prior or later describe embodiment reducing devices to facilitate a better grip of the stabilization member or implant head.

FIG. 10 illustrates an embodiment of the present invention with a ratchet mechanism 110 to adjustably lock the position of the second jaw 40 with respect to the first jaw 16. The ratchet mechanism 110 has a paw 112 that by gravity or by a spring loading can be torsionally biased to engage with a rack 114. The ratchet mechanism 110 can be used alone or in conjunction with the movement mechanisms should described in FIGS. 5, 7, 8 and 9.

FIG. 11 illustrates a first jaw 16 with a tapered opening 128 to allow the jaw 16 to align more closely with the implant head or with a screw thread of the threaded portion 24 of the pedicle screw.

The embodiment of the present invention shown in FIGS. 5 to 10 can also utilize a second jaw 48 as shown in FIGS. 3 and 4. While preferred embodiments of the present invention have been disclosed, it is to be understood it has been described by way of example only and that various other modifications can be made to the present invention without departing from the spirit and scope of the invention as it is encompassed in the following claims.

1. A device for reducing a stabilization member with a medical implant having a head and bone engaging portion comprising:
a first jaw for engaging said implant underneath said implant head;
a second jaw for engaging said stabilization number, said second jaw being slideably mounted with respect to said first jaw and being movable with respect to said first jaw to reduce said stabilization member with said head of said implant.

2. A device as described in claim 1 wherein said first jaw is linearly movable with respect to said first jaw.

3. A device as described in claim 1 wherein said device can be utilized with an elongated rod stabilization member.

4. A device as described in claim 1 wherein said device can be utilized with a plate stabilization member.

5. A device as described in claim 3 wherein said plate stabilization member has a slot that said implant head extends through.

6. A device as described in claim 1 wherein a rack and pinion mechanism moves said jaws with respect to one another.

7. A device as described in claim 1 wherein a jackscrew mechanism moves said jaws with respect to one another.

8. A device as described in claim 1 wherein a ratchet mechanism locks a position of said jaws with respect to one another.

9. A device as described in claim 1 wherein a lever mechanism moves said jaws with respect to one another.

10. A device as described in claim 9 wherein said lever mechanism includes a two bar linkage.

11. A device as described in claim 9 wherein said lever mechanism includes a pin in slot mechanism.

12. A device as described in claim 1 wherein said implant has a head with posts juxtaposed by a rod receiving area

13. A device as described in claim 1 wherein said first jaw has a slot allowing said implant to juxtapose opposing portions of said first jaw.

14. A device as described in claim 1 wherein said second jaw has a slot allowing said implant head to juxtapose opposing portions of said first jaw.

15. A device as described in claim 1 wherein said second jaw has a slot allowing said implant head to juxtapose opposing portions of said second jaw.

16. A device as described in claim 1 wherein said second jaw has an indention for receiving a portion of said stabilization member.

17. A device as described in claim 1 wherein said second jaw has an indention for receiving a portion of said implant head.

18. A device as described in claim 1 wherein said first and second jaws have portions joined in a tongue and groove type connection.

19. A device for reducing a stabilization member with a medical implant having a head and bone engaging portion comprising:
a first jaw for engaging said implant underneath said implant head;
a second jaw for engaging said stabilization number, said second jaw being slideably mounted with a tongue and groove connection with said first jaw and being movable with respect to said first jaw to reduce said stabilization member with said head of said implant

20. A method for reducing a stabilization member with a medical implant having a head and bone engaging portion comprising:
engaging said implant underneath said implant head with a first jaw;
engaging said stabilization number with a second jaw being slideably mounted and movable with respect to said first jaw to reduce said stabilization member with said head of said implant.
